(19) United States
(12) Patent Application Publication
(10) Pub. No.: US 2011/0008205 A1
(43) Pub. Date: Jan. 13, 2011

(54) MULTIFUNCTIONAL FLOOR PADS

(76) Inventor: John R. Mangiardi, Maur (Zurich) (CH)

Correspondence Address: DECHERT LLP, P.O. BOX 390460, MOUNTAIN VIEW, CA, 94039-0460, US

(21) Appl. No.: 12/160,370

(22) PCT Filed: Jul. 20, 2006
(86) PCT No.: PCT/US2006/028227

§ 371 Date: Aug. 27, 2010

Related U.S. Application Data
(60) Provisional application No. 60/758,638, filed on Jan. 14, 2006.

(51) Int. Cl. A61L 2/10

(52) U.S. Cl. 422/23; 422/24; 250/434

(57) ABSTRACT

A method of sterilizing a hospital room to at least 99.99% sterility is described which employs UV/ozone sterilization. A method of sterilizing sink-traps using UV radiation is described. A method utilizing UV/ozone sterilization and sink-trap sterilization by UV radiation for sterilization of an environment is also described.

### CROSS-REFERENCE TO RELATED APPLICATIONS

This application claims the benefit of provisional patent application U.S. Ser. No. 60/758,638 filed Jan. 14, 2006 by the present inventor. The contents of U.S. Ser No. 60/758,638 are expressly incorporated herein by reference thereto.

The following references are hereby explicitly incorporated by reference thereto:
U.S. Pat. No. 5,086,692
U.S. Pat. No. 6,656,424
U.S. Pat. No. 6,911,177
Applications filed along with present application by current inventor on this date entitled:
   IN-CEILING FOCUS LOCATED SURGICAL LIGHTING
   HOSPITAL OPERATING ROOM RE-DESIGN
   AMBIENT LIGHTING IN HOSPITAL SURGICAL ENVIRONMENTS
   IN-WALL WASTE RECEPTACLES FOR HOSPITAL AND LABORATORY ENVIRONMENTS
   MULTIFUNCTIONAL FLOOR PODS
   RE-DESIGN OF OPERATING ROOM TABLES
   ROBOTIC FLOOR CLEANING WITH STERILE, DISPOSABLE CARTRIDGES

### BACKGROUND OF THE INVENTION

1. Field of Invention

The present invention relates to devices and methods for producing sanitary health care environments, such as the operating room or other ambulatory treatment facilities, by utilizing ultraviolet irradiation and ozone to destroy viruses, bacteria, and fungus.

2. Background of the Invention

Hospital-acquired infections are responsible for tens of thousands of fatalities every year. These nosocomial infections are especially difficult to treat since increasingly, the strains are drug resistant. For example, it is estimated that 50% of staphylococcal strains (which can cause infection in post-op incisions) are resistant to all antibiotics currently in use. One approach to the control of such infectious agents involves the use of ultraviolet radiation in the “C” band range of around 200-280 nm. This technique is known as ultraviolet germicidal irradiation (UVGI). It is not a new technique, having been used as early as 1909 to disinfect the municipal water supply of Marseilles, France. More recently, it has been used to control contamination of air handlers and in isolation-rooms, especially for tuberculosis patients in hospitals. Other sterilization techniques include using ozone, but the ability to use ozone is limited because exposure to ozone is unhealthful for humans. Further, many decontamination means are incapable of providing a truly sterile environment. For example, while water-purifiers exist for water coming into a hospital sink, there is no effective method for sterilizing sink-traps. Sink-traps are a major source of drug-resistant, dangerous biologics. Hospitals are undertaking extensive infection-control programs. The operating room should be an active part of such efforts; therefore, a method and/or apparatus that provides a sterile hospital room environment would be of benefit.

UVGI can be utilized in the operating room environment to help sterilize contaminations from a variety of sources including infected equipment brought in, the surgical patient, the surgical team, and outside air.

As contaminants are brought into contact with UVC-band radiation, both the membrane and nucleus are penetrated. The UVC-band light then breaks up the molecular bonds of the DNA of the microorganism, thereby killing the microbe or inhibiting its reproduction. Spores and some bacteria tend to be somewhat more resistant than viruses, but all succumb to some dose of radiation after a period of time. Further, organic compounds which are exposed to UVC-band radiation are placed in an excited-energy state. In combination with a reactive molecule such as ozone, the excited organic compound or organism is more likely to react, leading to the destruction of the compound/organism, usually through an oxidative pathway. Ozone has been shown to be an effective sterilizer but is a strong irritant and unhealthy for humans. As such, an invention that can effectively combine ozone and UV sterilization while minimizing exposure to humans would provide a means for a sterile environment.

One source of contamination in the operating room environment is water from brought in for use in sinks. While there are numerous UV water purification systems, there is no known UVGI system for maintaining the sterility of a water trap. While some laboratory environments will utilize bleach or other sanitizers to kill contaminants in a sink trap, these methods require constant re-sterilization and are not sufficient for hospital environments where even sporadic infection of a sink trap can be dangerous. These pathogens can breed indiscriminately within a hospital sink trap and escape to the ambient air by advancing rearward and upward from the u-shaped sink trap to the water outlet area in a sink, and then to the hospital ambient air. The pathogens can then be carried by hospital workers, patients, movable equipment, and circulating air conduit systems. Since resistant, deadly organisms are harbored in hospital sink traps, a device and method that maintains constant sterility of a sink trap would be of benefit.

The survival probability of bacteria after being exposed to UVGI depends both on the irradiance as well as the exposure time in the general form of the following formula:

% Survival=100×e^{kIt} (1)

where in formula (I):
e=Napier's constant, approximately equal to 2.7183 and defined such that the natural logarithm of e is one.
I=UV irradiance in microwatts per square centimeter:

$$\frac{μ W}{{cm}^{2}}$$

t=time of UV exposure in seconds
k=microbe susceptibility factor in square centimeters per microwatt seconds:

$$\frac{{cm}^{2}}{μ W} \times s$$

It is the “k” factor that differentiates the irradiation time necessary to kill a particular microorganism. The prior art utilizes UV irradiation to provide a lethal dose of radiation and requires hospital personnel to leave the environment in which sterilization is occurring. Since a single organism could require a lengthy dose of radiation, because of a low k factor, proper use of such devices may involve the room being vacant, and hence unusable, for a long period of time.

Current devices, such as the air-handling system of Welch in U.S. Pat. No. 5,086,692 or the UV room sterilizer of Deal in U.S. Pat. No. 6,911,177, provide more sanitary or sterile areas of the hospital room, but cannot reach all spaces even with reflected UV rays. For example, cabinetry surfaces that are not in the line of sight of a directed UV emission or reflected UV emission will not sterilize. With the necessity that hospital operating room environments be as sterile as possible, a device that can provide at least 99.99% sterility on all exposed surfaces in a hospital room would be of benefit. A room is 99.99% sterile when 99.99% of all (previous to sterilization) known pathogens are destroyed. A pathogen is any fungus, virus, or bacterium typically found in hospital room environments.

It is an object of this invention to provide a sanitizing device for a sink-trap which utilizes UV rays to destroy pathogens residing within said sink-trap,

It is another object of this invention to provide a method for sanitizing a hospital operating room to at least 99.99% sterility,

It is yet another object of this invention to provide an integrated method for sanitizing a hospital operating room and improving the efficiency of maintaining the sterility thereof.

### SUMMARY OF THE INVENTION

The operating room or other health care environment of this invention uses UVGI to control contamination in a hospital room environment. Sanitization is provided by a ceiling-mounted UV/ozone sterilizer and a sink trap sterilizer. These devices, optionally used in combination with other traditional devices such as an air-duct sanitizer or floor sanitizer, provide means for an improved method of room sanitation. The sink-trap sanitizer, in addition to any air-handling devices, can be used continuously. These devices can also provide full-room sanitation for when operating room personnel are not present when used in combination with an airtight operating room.

The ceiling device utilizes UV tubes to provide an irradiation source and an ozone generator to provide ozone. More than one tube is provided on each device and the tubes are segregated into an up region and a down region. The up region irradiates ceiling and wall surfaces in its light of sight and surfaces reached by reflection. The down region is designed to irradiate all other surfaces within its line of sight and those that are reached by reflection of UV rays. When used, the room is evacuated of personnel. Ozone is generated during the emission of UV light. The UV light and ozone work alone and synergistically to destroy pathogens. Particularly, the ozone is able to reach non-reflected surfaces because of its gaseous nature.

Other areas such as sink trap wells are sterilized by a separate device that irradiates the sink-trap well. One embodiment places the UV source within the sink-trap, whereas another embodiment provides a UV transparent sink-trap portion and an outside UV source placed near the sink trap transparent portion. The above devices may be used in conjunction with other known sterilization means, such as an air duct sanitizer or a water-source sanitizer to provide an improved method of room sterilization.

### BRIEF DESCRIPTION OF THE DRAWINGS

The present invention can best be understood in connection with the accompanying drawings. It is noted that the invention is not limited to the precise embodiments shown in drawings, in which:

FIG. 1 is a perspective view of one possible embodiment of the UV/ozone, ceiling-mounted sterilizer.

FIG. 2 is a perspective view of a modified sink-trap containing a sink-trap sterilizer.

### BRIEF DESCRIPTION OF REFERENCE NUMERALS

100 ceiling mounts; 102 UV tube; 104 ozone generator; 106 room sterilizer; 200 sink basin; 202 sink drain; 204 piping; 206 sink-trap piping; 208 power cord; 210 UV bulb; 212 gasket; 214 bulb compartment

### DETAILED DESCRIPTION OF THE INVENTION

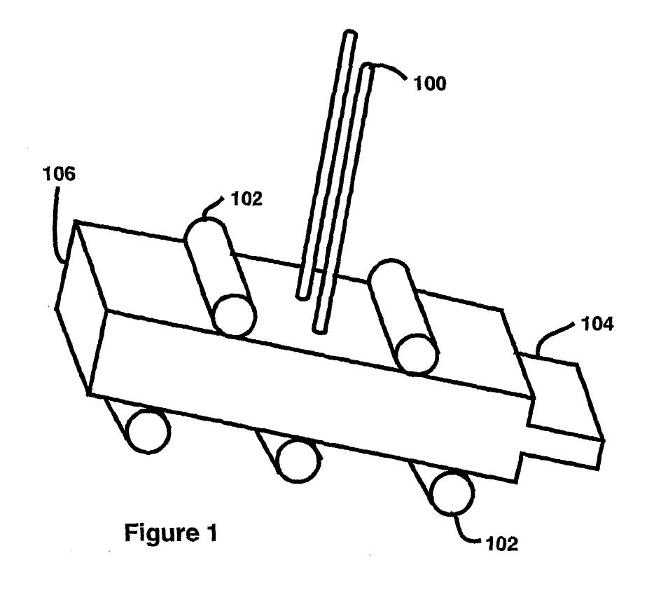

FIG. 1 is a perspective view of one possible embodiment of the UV/ozone, ceiling-mounted sterilizer. When the device is mounted within an operating room as by mounts 100, preferably on a ceiling, and the airtight room evacuated of personnel, it is powered on. By providing emission of UV rays from the UV bulbs 102 in conjunction with ozone from the ozone generator 104, the room sterilizer 106 sterilizes the room. The method is superior to UV only sterilization techniques because the production of ozone sterilizes surfaces not in the line of sight of direct or reflected UV rays. Further, the method is used in conjunction with an airtight room, thereby allowing high concentrations of ozone to be generated. After a sufficient period of time has elapsed, the generator may be turned of, such as by an outside the room power switch. Ozone decomposes naturally into harmless components; therefore, after a suitable waiting period, the room is sterilized to at least 99.99% sterility and personnel may reenter.

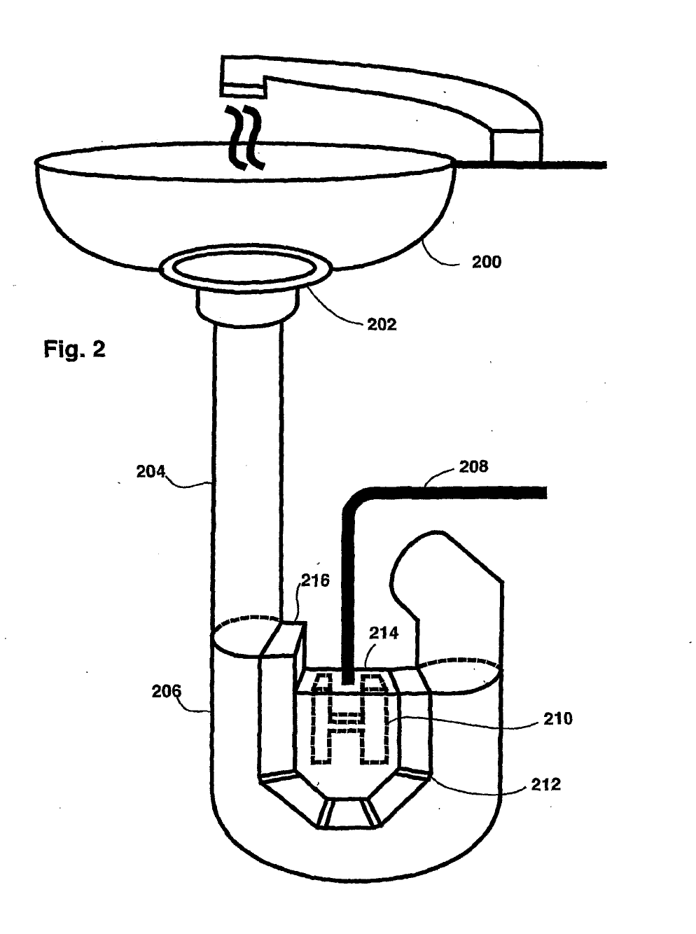

FIG. 2 shows a typical embodiment for purifying hospital sink-trap pipe-sections with ultraviolet irradiation.

In FIG. 2, the drain sink-trap pipe-section 204 leading from the sink drain 202 in sink basin 200 is interrupted by a plurality of flat transparent quartz glass windows 216. These windows may be flat, thick fused quartz windows, which are sealed by silicone seal gaskets 212 and bonded to the trap pipe section 206. Adjacent to the windows 216 there is provided a UVGI source 210, such as a PL-LI8 W/TUV, 18 W 2 GII base lamp, such as manufactured by “Topbulb Company”, within an opaque housing 214 enclosing UV lamp 210. Housing 214 has a UV reflective internal surface within the housing 214. Housing 214 is preferably aluminum with cast external fins (not shown) to dissipate heat. It is noted that while FIG. 2 show U-shaped pipe sections of a sink drain trap, it is further noted that the entire sink drain can also be formed of the transparent quartz material, as opposed to the preferable embodiments having sections thereof.

In the foregoing description, certain terms and visual depictions are used to illustrate the preferred embodiment. However, no unnecessary limitations are to be construed by the terms used or illustrations depicted, beyond what is shown in the prior art, since the terms and illustrations are exemplary only, and are not meant to limit the scope of the present invention. It is further known that other modifications may be made to the present invention, without departing the scope of the invention, as noted in the appended claims.

1. A method of sterilizing a room until sterilized 99.99% or greater, comprising:
providing a room sterilizer comprising
at least one source of ultraviolet radiation, substantially in the UV C band range, and
at least one source of ozone;
providing a room that is substantially air-tight;
evacuating personnel from the room;
emitting said ultraviolet radiation and said ozone until room is sterilized 99.99% or greater.

2. The method of claim 1 in which said room sterilizer has a detector adapted to sense the presence of persons within the room and further adapted to prevent functioning of said room sterilizer when said persons are within the room.

3. The method of claim 1 in which said room sterilizer has an alarm adapted to sound when personnel are in the room and the room sanitizer is on or there exists dangerous levels of ambient ozone.

4. The method of claim 1 in which a sink trap is provided, said sink-trap adapted to be sterilized by UV C band range radiation, and in which said sink trap is then continuously sterilized by UV C band range radiation.

5. A method of sterilizing a sink trap, comprising:
providing a sink-trap containing
sink-trap piping adapted with UV transparent materials;
providing a source of UV radiation substantially in the UV C band range;
emitting UV radiation from said source into said sink-trap.

6. The method of claim 5 in which UV radiation is emitted continuously.

7. A sink-trap sterilizer, comprising:
a source of ultraviolet radiation, substantially in the UV C band range;
a sink-trap pipe adapted to allow UV C band radiation to pass through the pipe material;
a power source adapted to power said source of ultraviolet radiation;
a housing enclosing said source of ultraviolet radiation.

8. The sink-trap sterilizer of claim 7 in which said housing is adapted to dissipate heat

9. The sink-trap sterilizer of claim 8 in which said housing is adapted to dissipate heat by being composed of metal

10. The sink-trap sterilizer of claim 9 in which said metal is aluminum.

11. The sink-trap sterilizer of claim 7 in which said housing has an internal reflective surface.

12. The sink-trap sterilizer of claim 7 in which said sink-trap pipe material is made of quartz.

13. A method of claims 1 further comprising:
providing a sink-trap containing sink-trap piping adapted with UV transparent materials;
providing a source of UV radiation substantially in the UV C band range;
continuously emitting UV radiation from said source into said sink-trap.

14. The method of claim 2 in which said room sterilizer has an alarm adapted to sound when personnel are in the room and the room sanitizer is on or there exists dangerous levels of ambient ozone.

15. The sink-trap sterilizer of claim 8 in which said housing has an internal reflective surface.
